(19) United States
(12) Patent Application Publication
(10) Pub. No.: US 2025/0325402 A1
(43) Pub. Date: Oct. 23, 2025

(54) MICRONEEDLE DEVICE FOR INTRAOCULAR INJECTIONS AND METHODS OF USING THE SAME

(71) Applicant: OCULOGENEX INC., La Habra, CA (US)

(72) Inventors: Hema Lakshmi Ramkumar, Seal Beach, CA (US); Ramaswamy Ramkumar, Whittier, CA (US)

(73) Assignee: OCULOGENEX INC., La Habra, CA (US)

(21) Appl. No.: 19/197,886

(22) Filed: May 2, 2025

Related U.S. Application Data
(63) Continuation of application No. PCT/US2025/016957, filed on Feb. 22, 2025.
(60) Provisional application No. 63/557,407, filed on Feb. 23, 2024.
(60) Provisional application No. 63/637,299, filed on Apr. 22, 2024.

(51) Int. Cl. A61F 9/00; A61M 5/32; A61M 5/34

(52) U.S. Cl. CPC A61F 9/0017; A61M 5/3286; A61M 5/348

(57) ABSTRACT

A needle holding unit including a needle, a needle base attached to the needle, a needle hub attached to the needle base where the top surface of the needle is a contoured surface extending from a high point to a low point. The needle is used to inject into an eye, including for suprachoroidal injection and intraocular injection.

### BACKGROUND OF THE PRESENT INVENTION

Intraocular injections are used to deliver drug therapy to the back of the eye. Intraocular injections are used to treat a number of retinal diseases. To be effective, the injection must be highly controlled and must deliver medication to a specific area in a small space and at a specific depth. For that reason, the size and configuration of the needle is critical to the success of the injection.

Due to the complexity of intraocular injections, standard needles are not properly configured to deliver medication between the sclera and the choroid, the suprachoroidal space. The use of incorrectly configured needles can result in the improper distribution of the medication reducing the effectiveness of the treatment.

Currently, suprachoroidal (SC) route of administration (ROA) allows for in-office, rather than surgical delivery, with no reported cases of vision loss, and a greater durability of gene therapies (2+ years). As a result, it eliminates the need for frequent intraocular injections. More particularly, SC delivery via the Clearside device of an investigational gene therapy reduced the frequency of treatments by 80%, with 50% of patients no longer requiring treatment. However, there were side effects. For instance, 18-30% of patients who received SC delivery developed episcleritis, a painful condition requiring steroid treatment. Though the exact cause of episcleritis is not known, it is possible that the high episcleritis rate is due to AAV efflux in the episcleral space due to a larger diameter (30G) needle and longer bevel design. The device used is effective for secreted molecules but has limited penetration in the posterior pole. Everads (Israel) has an investigational device with a larger diameter (27/30G) that utilizes a dissecting wire with potential for scarring that could prevent future redosing. Both devices (Clearside and Everads) have a 70-80 μL dead space, resulting in drug waste and potential underdosing when treatment volumes are only 100 μl. While a combination SC device and treatment (Clearside Xipere14) is FDA-approved for the treatment of uveitis, no FDA-cleared stand-alone SC delivery device is available for genetic treatment of macular diseases, resulting in a bottleneck in the utilization of this ROA. Therefore, a need exists for a needle that will allow for the proper insertion and distribution of medication into the suprachoroidal space to deliver therapeutics to the back of the eye.

The present invention provides for a novel ophthalmic suprachoroidal delivery device (SCDD) that allows for efficient and effective drug delivery for use in patients and for clinical application. The present invention provides for a design and manufacturing process that will work for ophthalmic applications.

### SUMMARY OF THE PRESENT INVENTION

One embodiment of the present disclosure may include a needle holding unit having a needle, a needle base attached to the needle, a needle hub attached to the needle base, where the top surface of the needle is a contoured surface extending from a high point to a low point.

In another embodiment, the contoured surface may have an angle relative to a vertical back wall of between 22 and 45 degrees.

In another embodiment, the needle hub may include a locking unit to lock the needle hub to a syringe.

In another embodiment, the contoured surface may have an angle relative to a vertical back wall of between 22 and 45 degrees.

In another embodiment, the locking unit may be configured to engage a Luer lock on the syringe.

In another embodiment, the locking unit may be configured to engage a Catheter lock on the syringe.

In another embodiment, the needle may include an opening in the contoured cutting surface with the opening connecting to a channel in the needle that allows fluid to flow.

In another embodiment, the lower point of the contoured cutting surface may be connected to a lower portion by an offset.

In another embodiment, the offset may be angled towards the backwall and connects to the low point of the contoured cutting surface.

In another embodiment, the needle may have a length of 900 microns and 1100 microns.

In a further embodiment, the needle may have a length of 200 microns, 300 microns, 400 microns, 500 microns, 600 microns, 700 microns, 800 microns, 900 microns, 925 microns, 950 microns, 975 microns, 1000 microns, 1025 microns, 1050 microns, 1075 microns, 1100 microns, 1200 microns, 1300 microns, 1400 microns or 1500 microns.

Another embodiment of the present disclosure may include a method of forming a needle holding unit including steps of forming a needle, attaching a needle base to the needle, and attaching a needle hub to the needle base, where the top surface of the needle is a contoured surface extending from a high point to a low point.

In another embodiment, the contoured surface may have an angle relative to a vertical back wall of between 22 and 45 degrees.

In a further embodiment, the contoured surface may have an angle relative to a vertical back wall of at least 10 degrees, 15 degrees, 20 degrees, 21 degrees, 22 degrees, 23 degrees, 24 degrees, 25 degrees, 26 degrees, 27 degrees, 28 degrees, 29 degrees, 30 degrees, 31 degrees, 32 degrees, 33 degrees, 34 degrees, 35 degrees, 36 degrees, 37 degrees, 38 degrees, 39 degrees, 40 degrees, 41 degrees, 42 degrees, 43 degrees, 44 degrees, 45 degrees, 46 degrees, 47 degrees, 48 degrees, 49 degrees, 50 degrees, 55 degrees, 60 degrees, 65 degrees, 70 degrees or 75 degrees.

In a further embodiment, the contoured surface may have an angle relative to a vertical back wall of no more than 10 degrees, 15 degrees, 20 degrees, 21 degrees, 22 degrees, 23 degrees, 24 degrees, 25 degrees, 26 degrees, 27 degrees, 28 degrees, 29 degrees, 30 degrees, 31 degrees, 32 degrees, 33 degrees, 34 degrees, 35 degrees, 36 degrees, 37 degrees, 38 degrees, 39 degrees, 40 degrees, 41 degrees, 42 degrees, 43 degrees, 44 degrees, 45 degrees, 46 degrees, 47 degrees, 48 degrees, 49 degrees, 50 degrees, 55 degrees, 60 degrees, 65 degrees, 70 degrees or 75 degrees.

In a further embodiment, the contoured surface may have an angle relative to a vertical back wall of about 10 degrees, 15 degrees, 20 degrees, 21 degrees, 22 degrees, 23 degrees, 24 degrees, 25 degrees, 26 degrees, 27 degrees, 28 degrees, 29 degrees, 30 degrees, 31 degrees, 32 degrees, 33 degrees, 34 degrees, 35 degrees, 36 degrees, 37 degrees, 38 degrees, 39 degrees, 40 degrees, 41 degrees, 42 degrees, 43 degrees, 44 degrees, 45 degrees, 46 degrees, 47 degrees, 48 degrees, 49 degrees, 50 degrees, 55 degrees, 60 degrees, 65 degrees, 70 degrees or 75 degrees.

Another embodiment includes the step of forming a locking unit on the needle base to lock the needle hub to a syringe.

In another embodiment, the locking unit may be configured to engage a Luer lock on the syringe.

In another embodiment, the locking unit may be configured to engage a Catheter lock on the syringe.

In a further embodiment, the lock is a slip tip or an eccentric tip lock.

In an embodiment, a syringe is an insulin syringe, a tuberculin syringe, a standard hypodermic needle, a safety syringe, a prefilled syringe, an auto-disable syringe, a plastic syringe, a glass syringe, a stainless steel syringe, safety syringe, Luer lip syringes, Catheter Tip syringes, oral syringe, gas syringe.

In an embodiment, a syringe is reusable. In another embodiment, a syringe is disposable.

Another embodiment may include the step of forming an opening in the contoured cutting surface with the opening connecting to a channel in the needle that allows fluid to flow.

In another embodiment, the lower point of the contoured cutting surface may be connected to a lower portion by an offset.

In another embodiment, the offset may be angled towards the backwall and connects to the low point of the contoured cutting surface.

In another embodiment, the needle may have a length of 900 microns and 1100 microns.

In another embodiment, the needle may have a length of 4 mm to 10 mm for intravitreal injection.

### FIGURE SUMMARY

A more complete appreciation of the invention and many of the attendant advantages thereof will be readily obtained as the same becomes better understood by reference to the following detailed description when considered in connection with the accompanying drawings, wherein:

FIG. 1 depicts a side view of a needle holding unit;

FIG. 2 depicts the top view of the needle in FIG. 1;

FIG. 3 depicts a perspective view of the needle of FIG. 1;

FIG. 4A depicts a side view of one embodiment of the needle of FIG. 1;

FIG. 4B depicts another embodiment of the needle of FIG. 1;

FIG. 4C depicts one embodiment of the needle of FIG. 1;

FIG. 4C depicts one embodiment of the needle of FIG. 1;

FIG. 4D depicts another embodiment of the needle of FIG. 1;

FIG. 5 depicts a syringe for use with the needle; and

FIG. 6 depicts the needle holding unit connected to the syringe.

FIG. 7 depicts a CAD rendering (left) and alpha prototype (right) of OCLX-SCDD.

FIG. 8 depicts pig eyes that were dissected, and representative photographs that demonstrated a clear vitreous without indocyanine green (ICG) dye (left), an intact retina (left) and RPE (middle), and circumferential and posterior temporal distribution (right) of the green ICG dye in the suprachoroidal space 1 h after SC injection with the Proof of Concept (POC) prototype #1 (POC1) of OCLX-SCDD (right).

FIG. 9 depicts ICG stained area (%) for OCLX-SCDD devices (POC1-POC4) compared to Clearside devices at the 1100 μm (CS1100) and 900 μm (CS900) lengths.

FIG. 10 depicts a CAD drawing of an OCLX-SCDD Proof of Concept (POC) prototype components in assembly (left) and dimensions (right). The exposed needle length is fully customizable from 700 to 1100 μm, depending on need.

FIG. 11 depicts a process capability sixpack report for needle length. With a sample size of n=33, needle length mean and SD obtained were 930.99±10.4 μm.

FIG. 12 depicts: (a) an infrared image (left) of the Yucatan minipig eye immediately after SC injection of ICG; (b) optical coherence tomography (OCT) image (middle) demonstrating hyperreflectivity (red arrow) in the suprachoroidal space, confirming drug delivery in targeted location with the OCLX-SCDD POC4 device; (c) Comparison of ICG-stained area 1 hour after SC injection and microdissection (right), demonstrating no significant difference between POC4 and the Clearside 1100 μm device.

FIG. 13 depicts: (a) Data demonstrating dose-dependent increase in retinal drug levels after SC injection. Suprachoroidal injection of OCLX-001 in doses of 1e¹¹, 5e¹¹ and 1e¹² vg/eye (low, medium and high, respectively) significantly increased drug protein levels compared with AAV8 stuffer in New Zealand white rabbits; (b) Schematic of ocular biodistribution after SC injection of OCLX-001 with OCLX-SCDD.

FIG. 14 depicts ocular tissue dissections demonstrating the suprachoroidal (SC) distribution of ICG dye (green) 1 hour after SC injection in Yucatan minipigs with OCLX-SCDD. The ICG dye was distributed throughout the temporal side, extending back to the posterior pole. Quantification of the green area resulted in 75% posterior pole ICG distribution at the 120 and 150 μl volumes.

FIG. 15 depicts a comparison of the ICG-stained area 1 hour after injection of different volumes in Yucatan minipig eyes with the POC4 OCLX-SCDD. ICG-stained areas of the whole suprachoroidal space (SCS) and temporal SCS are presented. Mean±SEM, ns: no significant difference.

FIG. 16 depicts intraocular pressure (IOP) changes over 1 day as measured in Yucatan minipigs (n=4) after injection with OCLX-SCDD. The slight increase in intraocular pressure at 1 minute normalizes after 15 minutes.

FIG. 17 depicts the successful delivery of AAV8 with OCLX-SCDD. One day after SCI with 1¹² vg/eye of an AAV8 viral vector, AAV8 viral genomes were quantified in Yucatan minipigs (n=4). A significant increase in AAV8 viral genomes was detected in all sections of the retina, including the posterior pole, at both 120 and 200 μl, compared to eyes treated with the same volumes of formulation buffer alone.

### DETAILED DESCRIPTION

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, the subject technology is not limited to the specific details set forth herein and may be practiced using one or more implementations. In one or more instances, structures and components are shown in simplified form in order to avoid obscuring the concepts of the subject technology. In the drawings referenced herein, reference numerals designate identical or corresponding parts throughout the several views or embodiments.

In an embodiment, an Oculogenex SC drug delivery device (OCLX-SCDD) provides a precise and controlled delivery of the drug directly to the SC space. In a further embodiment, the OCLX-SCDD minimizes episcleritis, improves therapeutic outcomes, and increases patient compliance.

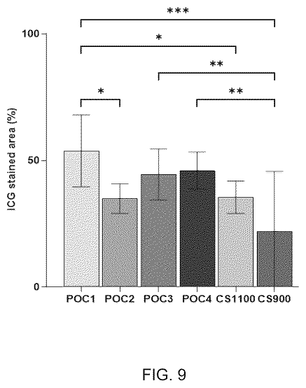

In an embodiment, the design of an OCLX-SCDD includes: (1) a smaller 33G needle for accurate targeting, (2) a short bevel seated deeper in the sclera to better target the SC space and avoid reflux, preventing underdosing and episcleral gene therapy exposure, (3) easy to use mechanism for more rapid surgeon uptake, (4) a Luer lock for compatibility with various syringes, and (5) a smaller dead space (e.g. 34 μl). Comparative studies showed 54% SC coverage in ex vivo pig eyes with OCLX-SCDD versus 22% using the Clearside device (FIG. 9). In vivo tests demonstrated 75% coverage of the temporal retina and posterior pole with OCLX-SCDD. Additionally, pharmacokinetic studies in rabbits (n=12) and minipigs (n=4) confirmed increased retinal drug levels with zero cases of episcleritis.

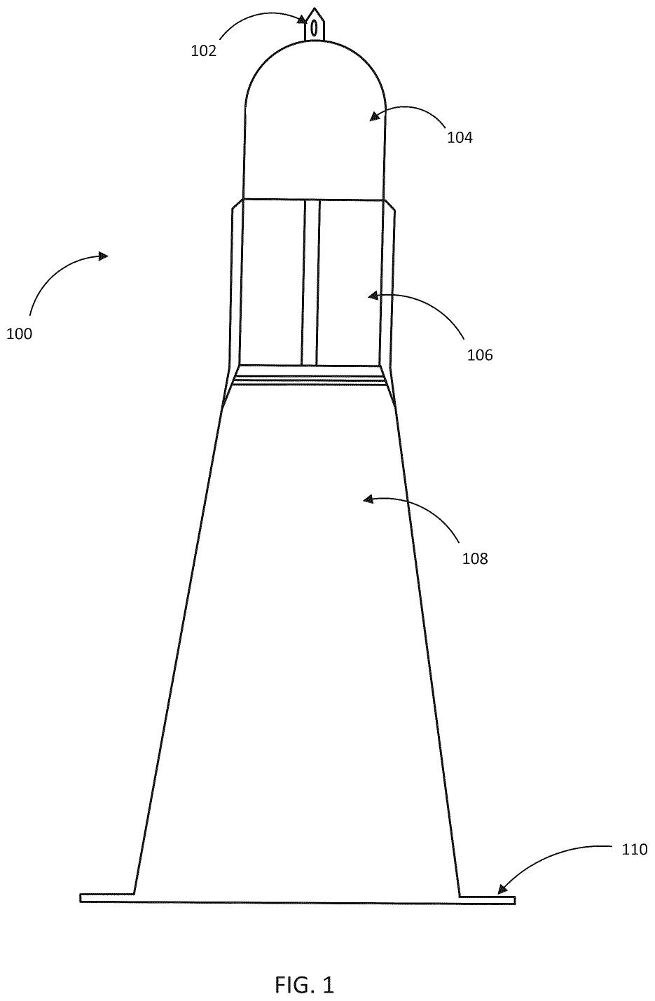

FIG. 1 depicts a side view of a needle holding unit 100. The needle holding unit 100 includes a needle 102 positioned on a top portion of a needle base 104 with the needle base 104 being attached to a needle hub 106. The needle 102 may be made of any suitable material including, but not limited to, stainless steel, tungsten and silicon. The bottom of the needle portion 108 of the needle hub 106 includes a ridge 110 that is configured to engage an opening of a needle barrel. In one embodiment, the needle 102 is 900 microns in height and the needle base 104 is made of silicone with a height of 4 mm. In another embodiment, the needle height is between 900 microns and 1100 microns and is 29-33 gauge. The needle hub 106 may be made of any suitably rigid material including, but not limited to, metal or plastic. The ridge 110 is configured to engage the grooves in a Luer lock on a needle base. In another embodiment, the ridge 110 is configured to engage a Luer slip on a needle body. In another embodiment, the ridge 110 is configured to engage a catheter syringe base. In another embodiment, the ridge 110 is configured to engage an oral tip on a syringe.

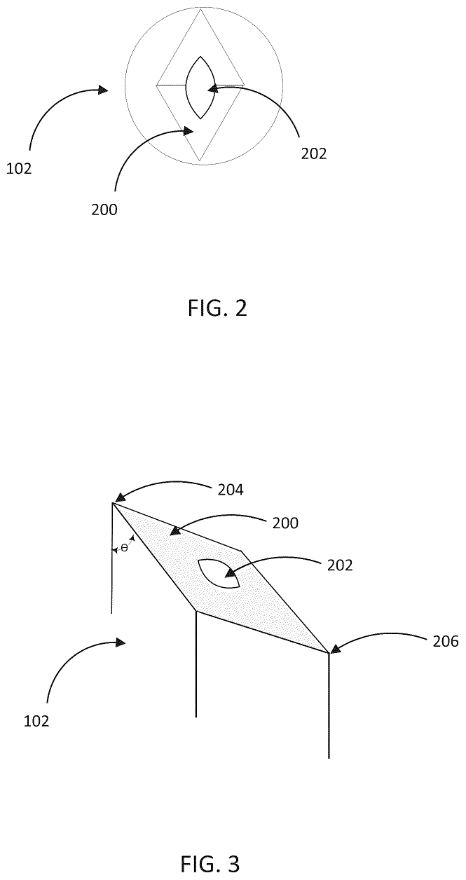

FIG. 2 depicts the top view of the needle 102. The needle 102 has a contoured cutting surface 200 to provide enhanced penetration into the suprachoroidal space of the eye. An opening 202 is formed through the needle 102 and into the base of the syringe to allow fluid to flow through the needle 102. FIG. 3 depicts a perspective view of the needle 102. The contoured cutting surface 200 is positioned at an angle theta (Θ) from vertical such that the contoured cutting surface 200 has a first point 204 that is higher relative to a second point 206. In one embodiment, the angle theta Θ is an angle between 22 degrees and 45 degrees. The opening 202 is formed in the contoured cutting surface 202 and is co-planar with the contoured cutting surface. The opening 202 is sized to allow for fluid to flow through the needle 102 and into a targeted region.

In one embodiment, the angle theta (Θ) is between 22 and 30 degrees. In another embodiment, the angle theta (Θ) is between 30 and 35 degrees. In another embodiment, the angle theta (Θ) is between 35 and 40 degrees. In another embodiment, the angle theta (Θ) is between 40 and 45 degrees. The opening 202 may be circular, oval, square, oblong or any other shape to allow for fluid flow.

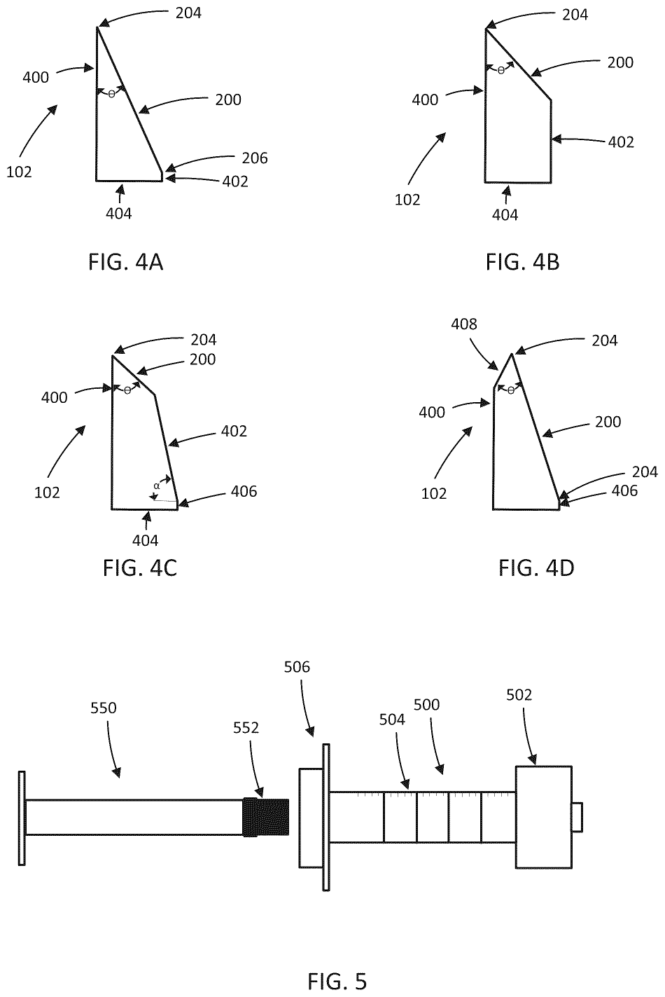

FIG. 4A depicts a side view of one embodiment of the needle 102. The needle 102 includes a back wall 400 connected to the high point 204 of the contoured cutting surface 200 to create the angle theta (Θ). The contoured cutting surface 200 slopes from a high point 204 to a lower point 206. An offset 402 connects the contoured cutting surface 200 to a lower wall 404. FIG. 4B depicts another embodiment of the needle 102. The backwall 400 is connected to the high point 204 of the contoured cutting surface 200 to form the angle theta (Θ). The offset 402 extends from the lower wall 404 with the offset 402 having length of at least half the length of the backwall 400.

FIG. 4C depicts one embodiment of the needle 102. The back wall 400 is connected to the high point 204 of the contoured cutting surface 200 to form the angle theta (Θ). An upper portion of the offset 402 is angled in relation to the lower wall 404 at an angle alpha (α) towards the low point 206 of the contoured cutting surface 200. FIG. 4D depicts another embodiment of the needle 102. The back wall 400 includes a tiled portion 408 that is connected to the high point 206 of the contoured cutting surface 200 to create the angle theta (Θ). The contoured cutting surface 200 extends from the high point 204 to the low point 206 on the top of the offset 406.

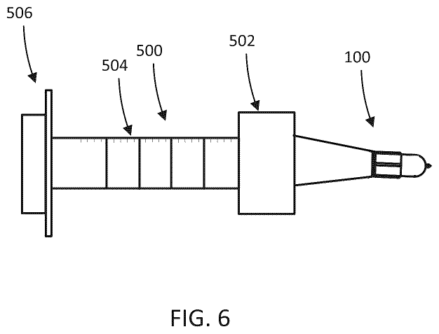

FIG. 5 depicts a syringe 500 for use with the needle 102. The syringe 500 includes a locking unit 502, a body 504 connected to the locking unit 502 and a base 506 connected to the body 504. The locking unit 502 may be any suitable locking unit 502 including, but not limited to, a Luer lock, a Luer slip or a catheter connection. The base 506 includes an opening that is connected to a channel that extends through the body 504 and locking unit 502 of the syringe 500. A plunger unit 550 including a sealing portion 552 is sized to engage the channel to push fluid through the channel. The sealing unit 552 is made of a formable material including, but not limited to, rubber, silicone or any other material capable of forming a seal with the inner sidewalls of the channel. The seal created by the sealing unit 552 is capable of drawing liquid through the needle 102 and into the channel. FIG. 6 depicts the needle holding unit 100 connected to the syringe. The channel running through the needle holding unit 100 is concentrically aligned with the channel in the syringe 500 body 504.

The needle of the present invention can be used to deliver a nucleic acid or a protein through an intraocular injection. The nucleic acid can be a DNA or an RNA, an analogue of a DNA or an RNA, a synthetic DNA or RNA. The RNA can be a full-length RNA encoding a protein, a tRNA, miRNA, snRNA, long non-coding RNA, mRNA, rRNA, or a circular RNA. A nucleic acid can be an artificial nucleic acid, including peptide nucleic acid, locked nucleic acid, morpholino nucleic acid, glycol nucleic acid and a threose nucleic acid. The DNA can be an A-DNA, B-DNA, C-DNA, D-DNA, E-DNA, Z-DNA, mitochondrial DNA, chloroplast DNA, The DNA can be a linear DNA, a plasmid. The DNA can be single-stranded, double-stranded or multi-stranded.

A protein can be a full-length protein or a peptide. The peptide can be a dipeptide, a tripeptide, an oligopeptide, for example a peptide with less than 20 amino acids or a polypeptide, for example, a peptide with more than 20 amino acids. The protein can be an antibody, a contractile protein, an enzyme, a hormonal protein, a structural protein, a storage protein or a transport protein. A protein can be from an animal, a plant, a bacteria, a virus or a parasite.

In an embodiment, the needle can be used to deliver cellular material (e.g. retinal progenitor cells, retinal pigment epithelial cells, or RPE/Bruchs membrane) for the treatment of retinal degenerative diseases.

In another embodiment, the needle can be used to deliver a viscoelastic material or hydrogel for the treatment of retinal detachments.

In a further embodiment, the needle can be used to deliver a therapeutic, including a small molecule (small chemical structure), a biologic (including, an antibody, a protein, a peptide, a bifunctional protein, a trifunctional molecule). The therapeutic can be used to treat a cancer, an autoimmune disease, type 2 diabetes, a cardiovascular disease and/or a kidney or liver disease.

In an embodiment, the protein is a polycomb complex protein BMI-1. In another embodiment, the DNA and/or RNA encodes a polycomb complex protein BMI-1.

The needle of the present invention can be used to deliver an AAV by intraocular injection.

The needle of the present invention can be used to inject into the back of the eye.

The needle of the present invention can be used to inject a therapeutic to treat a patient suffering from diabetic retinopathy, inherited retinal disease, macular degeneration, vein occlusion uveitis, macular telangiectasias, macular edema, dry eye, wet eye and/or age-related macular degeneration (AMD). In an embodiment, the therapeutic injected using the needle of the present invention is a polycomb complex protein BMI-1.

In an embodiment, the length of the needle is set to enhance the safety of injecting into an intravitreal space. In a further embodiment, the length of needle is set to optimize delivery of drugs into the intravitreal space. This is done by controlling the length of the needle that enters the mid-vitreous cavity. The needle can be a length of 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm, 50 mm, 51 mm, 52 mm, 53 mm, 54 mm, 55 mm, 56 mm, 57 mm, 58 mm, 59 mm, 60 mm, 61 mm, 62 mm, 63 mm, 64 mm, 65 mm, 66 mm, 67 mm, 68 mm, 69 mm, 70 mm, 71 mm, 72 mm, 73 mm, 74 mm, 75 mm, 76 mm, 77 mm, 78 mm, 79 mm, 80 mm, 81 mm, 82 mm, 83 mm, 84 mm, 85 mm, 86 mm, 87 mm, 88 mm, 89 mm, 90 mm, 91 mm, 92 mm, 93 mm, 94 mm, 95 mm, 96 mm, 97 mm, 98 mm, 99 mm, 100 mm or longer.

The needle can be a length of at least 1 mm, at least 2 mm, at least 3 mm, at least 4 mm, at least 5 mm, at least 6 mm, at least 7 mm, at least 8 mm, at least 9 mm, at least 10 mm, at least 11 mm, at least 12 mm, at least 13 mm, at least 14 mm, at least 15 mm, at least 16 mm, at least 17 mm, at least 18 mm, at least 19 mm, at least 20 mm, at least 21 mm, at least 22 mm, at least 23 mm, at least 24 mm, at least 25 mm, at least 26 mm, at least 27 mm, at least 28 mm, at least 29 mm, at least 30 mm, at least 31 mm, at least 32 mm, at least 33 mm, at least 34 mm, at least 35 mm, at least 36 mm, at least 37 mm, at least 38 mm, at least 39 mm, at least 40 mm, at least 41 mm, at least 42 mm, at least 43 mm, at least 44 mm, at least 45 mm, at least 46 mm, at least 47 mm, at least 48 mm, at least 49 mm, at least 50 mm, at least 51 mm, at least 52 mm, at least 53 mm, at least 54 mm, at least 55 mm, at least 56 mm, at least 57 mm, at least 58 mm, at least 59 mm, at least 60 mm, at least 61 mm, at least 62 mm, at least 63 mm, at least 64 mm, at least 65 mm, at least 66 mm, at least 67 mm, at least 68 mm, at least 69 mm, at least 70 mm, at least 71 mm, at least 72 mm, at least 73 mm, at least 74 mm, at least 75 mm, at least 76 mm, at least 77 mm, at least 78 mm, at least 79 mm, at least 80 mm, at least 81 mm, at least 82 mm, at least 83 mm, at least 84 mm, at least 85 mm, at least 86 mm, at least 87 mm, at least 88 mm, at least 89 mm, at least 90 mm, at least 91 mm, at least 92 mm, at least 93 mm, at least 94 mm, at least 95 mm, at least 96 mm, at least 97 mm, at least 98 mm, at least 99 mm, at least 100 mm or longer.

The needle can be a length of about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, about 50 mm, about 51 mm, about 52 mm, about 53 mm, about 54 mm, about 55 mm, about 56 mm, about 57 mm, about 58 mm, about 59 mm, about 60 mm, about 61 mm, about 62 mm, about 63 mm, about 64 mm, about 65 mm, about 66 mm, about 67 mm, about 68 mm, about 69 mm, about 70 mm, about 71 mm, about 72 mm, about 73 mm, about 74 mm, about 75 mm, about 76 mm, about 77 mm, about 78 mm, about 79 mm, about 80 mm, about 81 mm, about 82 mm, about 83 mm, about 84 mm, about 85 mm, about 86 mm, about 87 mm, about 88 mm, about 89 mm, about 90 mm, about 91 mm, about 92 mm, about 93 mm, about 94 mm, about 95 mm, about 96 mm, about 97 mm, about 98 mm, about 99 mm, about 100 mm or longer.

The needle can be a length of 10 microns, 20 microns, 30 microns, 40 microns, 50 microns, 60 microns, 70 microns, 80 microns, 90 microns, 100 microns, 110 microns, 120 microns, 130 microns, 140 microns, 150 microns, 160 microns, 170 microns, 180 microns, 190 microns, 200 microns, 210 microns, 220 microns, 230 microns, 240 microns, 250 microns, 260 microns, 270 microns, 280 microns, 290 microns, 300 microns, 310 microns, 320 microns, 330 microns, 340 microns, 350 microns, 360 microns, 370 microns, 380 microns, 390 microns, 400 microns, 410 microns, 420 microns, 430 microns, 440 microns, 450 microns, 460 microns, 470 microns, 480 microns, 490 microns, 500 microns, 510 microns, 520 microns, 530 microns, 540 microns, 550 microns, 560 microns, 570 microns, 580 microns, 590 microns, 600 microns, 610 microns, 620 microns, 630 microns, 640 microns, 650 microns, 660 microns, 670 microns, 680 microns, 690 microns, 700 microns, 710 microns, 720 microns, 730 microns, 740 microns, 750 microns, 760 microns, 770 microns, 780 microns, 790 microns, 800 microns, 810 microns, 820 microns, 830 microns, 840 microns, 850 microns, 860 microns, 870 microns, 880 microns, 8900 microns, 900 microns, 910 microns, 920 microns, 930 microns, 940 microns, 950 microns, 960 microns, 970 microns, 980 microns, 990 microns, 1000 microns, 1010 microns, 1020 microns, 1030 microns, 1040 microns, 1050 microns, 1060 microns, 1070 microns, 1080 microns, 1090 microns, 1100 microns, 1110 microns, 1120 microns, 1130 microns, 1140 microns, 1150 microns, 1200 microns or longer.

The needle can be a length of at least 10 microns, at least 20 microns, at least 30 microns, at least 40 microns, at least 50 microns, at least 60 microns, at least 70 microns, at least 80 microns, at least 90 microns, at least 100 microns, at least 110 microns, at least 120 microns, at least 130 microns, at least 140 microns, at least 150 microns, at least 160 microns, at least 170 microns, at least 180 microns, at least 190 microns, at least 200 microns, at least 210 microns, at least 220 microns, at least 230 microns, at least 240 microns, at least 250 microns, at least 260 microns, at least 270 microns, at least 280 microns, at least 290 microns, at least 300 microns, at least 310 microns, at least 320 microns, at least 330 microns, at least 340 microns, at least 350 microns, at least 360 microns, at least 370 microns, at least 380 microns, at least 390 microns, at least 400 microns, at least 410 microns, at least 420 microns, at least 430 microns, at least 440 microns, at least 450 microns, at least 460 microns, at least 470 microns, at least 480 microns, at least 490 microns, at least 500 microns, at least 510 microns, at least 520 microns, at least 530 microns, at least 540 microns, at least 550 microns, at least 560 microns, at least 570 microns, at least 580 microns, at least 590 microns, at least 600 microns, at least 610 microns, at least 620 microns, at least 630 microns, at least 640 microns, at least 605 microns, at least 6600 microns, at least 67 microns, at least 680 microns, at least 690 microns, at least 700 microns, at least 710 microns, at least 720 microns, at least 730 microns, at least 740 microns, at least 750 microns, at least 760 microns, at least 770 microns, at least 780 microns, at least 790 microns, at least 800 microns, at least 810 microns, at least 820 microns, at least 830 microns, at least 840 microns, at least 850 microns, at least 860 microns, at least 870 microns, at least 880 microns, at least 890 microns, at least 900 microns, at least 910 microns, at least 920 microns, at least 930 microns, at least 940 microns, at least 950 microns, at least 960 microns, at least 970 microns, at least 980 microns, at least 990 microns, at least 1000, 1010 microns, 1020 microns, 1030 microns, 1040 microns, 1050 microns, 1060 microns, 1070 microns, 1080 microns, 1090 microns, 1100 microns, 1110 microns, 1120 microns, 1130 microns, 1140 microns, 1150 microns, 1200 microns or longer.

The needle can be a length of about 10 micron, about 20 micron, about 30 micron, about 40 micron, about 50 micron, about 60 micron, about 70 micron, about 80 micron, about 90 micron, about 100 micron, about 110 micron, about 120 micron, about 130 micron, about 140 micron, about 150 micron, about 160 micron, about 170 micron, about 180 micron, about 190 micron, about 200 micron, about 210 micron, about 220 micron, about 230 micron, about 240 micron, about 250 micron, about 260 micron, about 270 micron, about 280 micron, about 290 micron, about 300 micron, about 310 micron, about 320 micron, about 330 micron, about 340 micron, about 350 micron, about 360 micron, about 370 micron, about 380 micron, about 390 micron, about 400 micron, about 410 micron, about 402 micron, about 430 micron, about 440 micron, about 450 micron, about 460 micron, about 470 micron, about 480 micron, about 490 micron, about 500 micron, about 510 micron, about 520 micron, about 530 micron, about 5400 micron, about 550 micron, about 560 micron, about 570 micron, about 580 micron, about 590 micron, about 600 micron, about 610 micron, about 620 micron, about 630 micron, about 640 micron, about 650 micron, about 660 micron, about 670 micron, about 680 micron, about 69 micron, about 700 micron, about 710 micron, about 720 micron, about 730 micron, about 740 micron, about 750 micron, about 760 micron, about 770 micron, about 780 micron, about 790 micron, about 800 micron, about 810 micron, about 820 micron, about 830 micron, about 840 micron, about 850 micron, about 860 micron, about 870 micron, about 880 micron, about 890 micron, about 900 micron, about 910 micron, about 920 micron, about 930 micron, about 940 micron, about 950 micron, about 960 micron, about 970 micron, about 980 micron, about 990 micron, about 1000 micron, 1010 microns, 1020 microns, 1030 microns, 1040 microns, 1050 microns, 1060 microns, 1070 microns, 1080 microns, 1090 microns, 1100 microns, 1110 microns, 1120 microns, 1130 microns, 1140 microns, 1150 microns, 1200 microns or longer.

In an embodiment, the needle is of a length that provides for the proper insertion and distribution of a therapeutic into the mid-vitreous space. In an embodiment, the therapeutic is delivered to the back of the eye of an individual or patient.

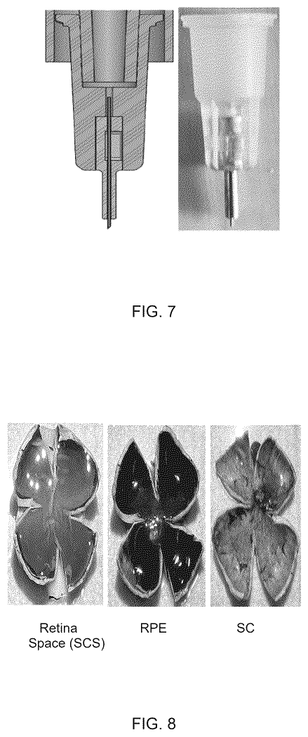

FIG. 7 shows an embodiment of an OCLX-SCDD of the present invention that overcomes limitations of current treatment methods. As depicted in FIG. 7, in this embodiment, the OCLX-SCDD features a series of innovative elements tailored to enhance drug delivery efficacy, safety, and patient compliance. It employs a smaller diameter needle to reduce tissue damage and discomfort, lower dead space than competitors-lowering the drug product volume needed to treat by more than 40%, reducing the possibility of underdosing and related reduced efficacy, while improving costs by reducing waste. This embodiment of the device also has an optimized shorter bevel that stays within the sclera to prevent drug reflux and related episcleritis and underdosing, and an innovative mechanism that simplifies the device's operation facilitating rapid adoption among healthcare providers. Furthermore, its compatibility with standard Luer lock syringes enhances its versatility and general usability with multiple drug vials, adapters, and prefilled syringes with the Luer lock.

By this embodiment of the device delivering medications directly to the suprachoroidal space without the limitations of current methods, the device of the present invention will enhance therapeutic outcomes and help to transform the management of chronic retinal diseases.

Experimental work has shown that there is no evidence of episcleritis when using the OCLX-SCDD, a common adverse event that results in patient pain and reduced compliance, which has been reported in another company's clinical studies. This invention contributes to patient quality of life, reduces healthcare costs by minimizing the frequency of treatments, and improves clinical outcomes in patients administered the therapeutic as part of the treatment. The development of OCLX-SCDD, in an embodiment, offers a significant advancement in medical technology and a shift of the retinal disease treatment paradigm by offering a safer, more effective, patient-friendly approach to delivering genetic therapies that can significantly reduce treatment burden by 80-100% and reduce adverse side effects associated with currently used treatment modalities.

In an embodiment, an OCLX-SCDD (see FIG. 7) provides a smaller diameter needle (for example, 30G, 31G, 32G, 33G, 34G, 35G, 36G, 37G, 38G or smaller), which minimizes tissue trauma, reduces infection and efflux risk, enhances patient comfort, and facilitates more precise and safer drug delivery directly within the suprachoroidal space. In an embodiment, an OCLX-SCDD has a shorter bevel design. The shorter bevel design, in an embodiment, is able to remain wholly embedded in the sclera during drug administration. This design feature prevents the common problem of drug reflux, ensuring full dosage delivery. This also maximizes treatment efficacy and safety. The low reflux design eliminates episcleritis, which can require systemic and topical therapy for months. Our low dead space design, (for example, 28 μl, 29 μl, 30 μl, 31 μl, 32 μl, 33 μl, 34 μl, 35 μl, 36 μl, 37 μl, 38 μl, 39 μl, 40 μl, 41 μl, 42 μl, 43 μl or more), lowers the drug product volume needed to treat, decreases costs with less product wasted, and prevents underdosing during delivery.

In an embodiment, an OCLX-SCDD incorporates an easy-to-use mechanism and simple tangential injection procedure that makes the device easier for surgeons to use. In an embodiment, the improvement found with the OCLX-SCDD has an integrated standard Luer lock that enhances the device's ability to be used by any surgeon or other person and its use with various therapeutics, including AAV therapeutics, proteins, peptides, nucleic acids, including RNA and DNA, across many different disease modalities. In an embodiment, an OCLX-SCDD can be used with a wide range of syringes.

In another embodiment, an OCLX-SCDD is manufactured using a polycarbonate (PC) outer hub and a stainless steel (SS) inner hub. The method of manufacturing can occur through machining, including, electrical discharge machining (EDM) to ensure precision and durability as well as through the use of molds, such as injection molding. In an embodiment, the two hubs are press-fitted together, with a strategically designed side glue pocket, that in one embodiment can be filled with Loctite 3974, a UV-curable adhesive, or any other adhesive, to strengthen the bond and enhance device stability.

In an embodiment, a delivery device, such as an OCLX-SCDD, is equipped with a single-bevel needle. In a further embodiment, a delivery device has a needle length of 925±30 μm. The alpha units include a makeshift cap, modified with a drilled hole to allow assembly testing while providing protection for the needle.

In another embodiment, each device, including an OCLX-SCDD, is individually packaged and labeled. In an embodiment, the package and label contain essential information, including, but not limited to, lot number, project number, usage guidance, and usage warnings (e.g., “not for human use”), along with the needle length and dead space range. In an embodiment a device, including an OCLX-SCDD, can be sterilized. In a further embodiment, sterilization is through the use of ethylene oxide (ETO) sterilization. Sterilization can ensure that the device has at a minimum, a baseline sterility and validate compatibility with a sterilization process.

In an embodiment, the device, including an OCLX-SCDD, has a tensile strength that provides for the safety, reliability, and effectiveness of the delivery device. Table 1 shows how the alpha design exceeds the 11N requirement by a good margin. Needle to hub tensile strength measured 49.4N+/−10.6N. Inner hub to Luer hub tensile strength measured 214.5N+/−18.5N. This connection must be strong enough to withstand the forces applied during injection. This includes the device, including an OCLX-SCDD, having the ability to penetrate tissue, such as the skin, the eye, or other surface of the patient. The tensile strength must be sufficient to deliver a viscous solution to the target tissue, including the eye. If the tensile strength is insufficient, a failure can or will occur including needle detachment or breakage during use. Such a failure leads to incomplete drug delivery, patient injury, or contamination of the drug or site of administration. In an embodiment, the device, including an OCLX-SCDD, has a robust needle-to-hub bond. The robust bond provides for consistent injection performance and maintains the device's integrity across repeated uses or under various handling conditions.

In an embodiment, a needle used with a device, including an OCLX-SCDD, has an exposed needle length (700 μm to 1100 μm) to ensure safe and effective delivery of therapeutics to the eye. In an embodiment, administration of the therapeutic into the eye is done at the posterior pole or vitreous cavity. In an embodiment, the chosen needle length ensures that injections reach only the correct anatomical location within the eye, maximizing therapeutic efficacy while minimizing the risk to the patient.

### Example 1

An 80-year-old woman with dry age-related macular degeneration is treated by injecting suprachoriodally an AAV8.BMI1, which encodes a polycomb complex protein BMI-1 protein using a needle of the present invention. Following injection, the woman begins to see a reduction in her symptoms related to her dry age-related macular degeneration and her eyesight improves.

### Example 2

A 40-year-old male with diabetes and retinopathy, who is suffering vision loss from macular edema, is treated by injecting suprachoriodally an AAV8.BMI1, which encodes a polycomb complex protein BMI-1 protein using a needle of the present invention. Following injection, the male begins to see a reduction in his symptoms related to his macular edema and his eyesight improves.

### Example 3

A 60-year-old male with Stargardt macular degeneration is treated by injecting suprachoriodally an AAV8.BMI1, which encodes a polycomb complex protein BMI-1 protein using a needle of the present invention. Following injection, the male begins to see a reduction in his symptoms related to his Stargardt macular degeneration and his eye site improves.

### Example 4

An 80-year-old woman with an advanced dry macular geographic atrophy is treated by injecting suprachoriodally an AAV8.BMI1, which encodes a polycomb complex protein BMI-1 protein using a needle of the present invention. Following injection, the woman begins to see a reduction in her symptoms and a slowing in the growth of the geographic atrophy.

### Example 5

A 55-year-old male with macular telangiectasias is treated by injecting suprachoriodally an AAV8.BMI1, which encodes a polycomb complex protein BMI-1 protein using a needle of the present invention. Following injection, the male begins to see a reduction in his symptoms related to his macular telangiectasias and his eyesight improves.

### Example 6

In an embodiment, the needle of the present invention can be used to inject a therapeutic intraocularly. In another embodiment, a therapeutic is a steroid, an antibody, a hormone, an antibiotic, an anti-VEGF antibody, an anti-complement antibody, a biologic therapeutic, and a small molecule therapeutic. In an embodiment, the length of the needle used to inject a therapeutic can be long, medium or short.

### Example 7

Ex vivo assessment of ocular biodistribution of ICG with OCLX-SCDD Proof of Concept (POC) prototypes and Clearside needles at 900 and 1100 μm in pig eyes.

In this non-GLP study, we evaluated the ability of OCLX-SCDD to deliver accurately to the suprachoroidal space (SCS) and determined the optimal needle length for effective delivery. Additionally, these tests examined the biodistribution of ICG dye within the ocular tissues to identify whether the 900 μm or 1100 μm needle length provides superior distribution. Ex vivo testing involved a comparative analysis of the dye's ocular biodistribution between OCLX-SCDD POCs 1-4 and the established Clearside device, allowing us to determine whether the new design offers improved delivery accuracy and effectiveness. Forty pig eyes were obtained from a farm 1 day prior to testing and SC 120 μl injections (5 mg/ml) were performed (one supertemporal injection site/eye) with ICG. Needles used included 900 μm and 1100 μm length samples from both Oculogenex and Clearside (CLS-900 and CLS-1100). One hour after injection, dissection of the eyes was performed. Images were taken with an iPhone 15 Pro camera after removal of retina, RPE/Choroidal tissue. The ICG-stained area was measured by a masked reader with Image-pro Plus 6 (Media Cybernetisc, Rockville, MD, USA) to objectively assess ICG distribution through software. The flow dynamics of suprachoroidal delivery with OCLX-SCDD in ex vivo porcine eyes demonstrated the spread of dye throughout the SCS (FIG. 8).

All (100%) of OCLX-SCDD prototypes successfully delivered ICG dye to the SCS alone with no undesired biodistribution to other ocular tissues. (2) POC1 had superior ICG uptake (54%) compared to CLS900 (22%) (p<0.05), and CLS1100 (36%) (p<0.001). POCs 3 (44%) and 4 (46%) had greater ICG biodistribution when compared to CLS 900 (FIG. 9).

### Example 8

In Vivo assessment of ocular biodistribution of ICG with the OCLX-SCDD POC prototype and Clearside needles at 1100 μm in pig eyes.

Suprachoroidal injections enable precise, targeted delivery to the retina, retinal pigment epithelium (RPE), and choroid, potentially offering higher bioavailability to the diseased tissues while minimizing impact on anterior segment tissues. To improve the outcome of gene therapy for AMD through the SCS, we tested if the POC4 SCDD results in ICG dye at the back of the eye. In this non-GLP study, the SC injection site was positioned 4 mm from the limbus in the superior temporal quadrant. This allows entrance into the suprachoroidal space in 4-month-old male minipigs (n=4). Indocyanine Green (ICG), 5 mg/ml, was injected using Clearside 1100 μm (OS) or POC4 of the OCLX-SCDD 1100 μm prototype device (OD) as follows: (1) Mark and confirm selected injection site; (2) Insert the microneedle, creating a small dimple on the ocular surface to allow the tissue to displace gently while the needle enters the suprachoroidal space; (3) Inject slowly and deliberately over 5 seconds to decrease discomfort; (4) After finishing the drug injection, hold the microinjector in place for an additional 3-5 seconds. Light pressure should be applied with a cotton swab after microinjector removal for about 5 seconds; and (5) Perform OCT imaging. (6) Euthanasia was performed according to the IACUC protocol, and 1 hour after injection, eyes were microdissected to assess ICG distribution.

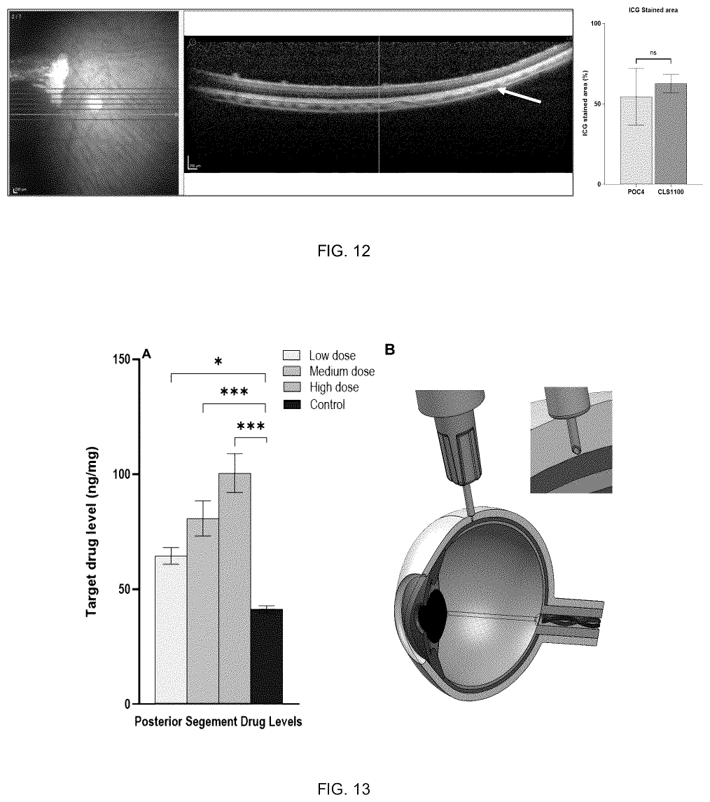

OCT imaging (FIG. 12) demonstrated successful suprachoroidal delivery. The ICG stained area was measured with Image-pro Plus 6 equipment. No significant differences were seen in the total coverage area (55%) with the Clearside and OCLX-SCDD POC devices when the 1100 μm needle lengths were compared. One of four eyes injected with the OCLX-SCDD 1100 μm POC prototype device had trace amounts of ICG in the vitreous. Based on the results in FIG. 12, to avoid intravitreal injection, alpha prototypes were redesigned to have a shorter length, targeting 925 μm, and future pig studies were performed with this needle length.

### Example 9

OCLX-SCDD pharmacokinetics/toxicokinetics (PK/TK) in vivo in rabbits.

A 12-week study in 3-month-old female New Zealand rabbits was conducted with 3 animals per group (n=12 total), bilaterally treated. Animals were treated with a posterior subtenon's injection of triamcinolone 20 mg at the time of injection.

Clinical examinations were performed throughout the study with serum and blood collected at the conclusion of the study. CBC, chemistry, and urine analysis were collected 3 months after treatment. Necropsy was performed at the conclusion of the study, and all organs were collected and analyzed for RNA and DNA analyses. The investigation used OCLX-001 at doses of 1¹¹ (the number of viral particles-a common notation for gene therapy), 5¹¹, 1¹² suprachoroidal injections (50 μl at each of two injection sites/eye). A suprachoroidal injection was performed 4 mm from the limbus for a volume of 100 μL with topical anesthetic (e.g., 0.5% proparacaine) instilled in each eye before the first injection. A 700 μm R&D version of the OCLX-SCDD was connected to a syringe containing the injection solution. The injection procedure (two injections, approximately superotemporally and inferonasally) consisted of passing the microneedle directly through the conjunctiva and the sclera at a point 4 mm posterior to the limbus. Upon injection, the fluid enters the suprachoroidal space. Following injection, erythromycin ointment was instilled on the ocular surface.

The retina and RPE were analyzed using an antigen-capture immunogenicity assay format on the MSD (Meso Scale Discovery, Rockville, MD) platform. Retina and RPE lysates were diluted to 5 μg/ml total protein concentration. Drug protein levels were detected by a custom developed MSD Meso QuickPlex SQ 120 MM assay using Discovery Workbench v. 4.0.

OCLX-001 increased RPE drug levels (1¹¹, 5¹¹, 1¹² vg/eye) in a dose-dependent fashion (FIG. 13A; 1e11, 5e11 and 1e12 doses yielded 1×, 2× and 2.4× control drug level in the RPE). ERG analyses demonstrated no evidence of retinal toxicity at any treatment dose. No cases of episcleritis or uveitis was noted in any animals, and no prophylactic oral steroids were administered.

### Example 10

OCLX-SCDD alpha prototype targeting of the SC space with ICG in pig eyes (FIG. 12).

Suprachoroidal injections enable precise, targeted delivery to the retina, retinal pigment epithelium (RPE), and choroid, potentially offering higher bioavailability to the diseased tissues while minimizing impact on anterior segment tissues. To improve SC gene therapy outcomes for AMD, we tested the alpha prototype device to determine whether the SCDD results in ICG dye distribution to the posterior pole. This study was conducted under non-GLP conditions, the SC injection site was positioned 4 mm from the limbus in the superior temporal quadrant, which allows entrance into the suprachoroidal space in 8-month old male minipigs. Indocyanine Green (ICG) (5 mg/ml), OS: 120 μl or OD: 150 μl, was injected using the OCLX-SCDD 925 μm alpha prototype as follows: (1) Mark and confirm selected injection site; (2) Insert the microneedle, being sure to create a small dimple on the ocular surface to allow the tissue to displace gently while the needle enters the suprachoroidal space; (3) Inject slowly and deliberately for 5 seconds; (4) After finishing the drug injection, hold the microinjector in place for an additional 5 seconds. Light pressure should be applied with a cotton swab after microinjector removal for about 5 seconds; and (5) Check IOP after injection. The eyes were then micro-dissected to check the injection site and ICG distribution immediately after injection.

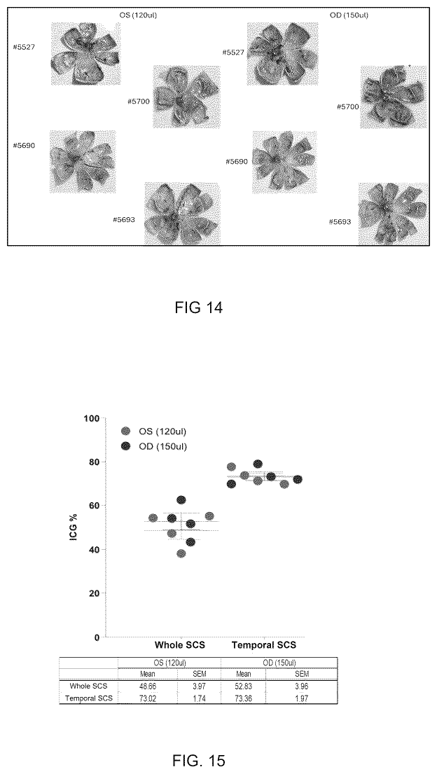

To evaluate the effect of SC injection, images were taken after removal of the retina and RPE/Choroidal tissue (FIG. 14). The ICG stained area was measured with Image-pro Plus 6. No significant differences were seen in the total coverage area (55%) and posterior pole coverage area (75%) when the 120 and 150 μl volumes were compared (FIG. 15). Based on the results obtained, the alpha prototypes have shown successful targeting of the SC space in 100% of Yucatan minipig eyes in vivo (n=8). Further, the ICG stained area showed reliability between 120 and 150 μl volumes in an in vivo study.

### Example 11

OCLX-SCDD alpha prototype and AAV8 targeting SC space in pigs.

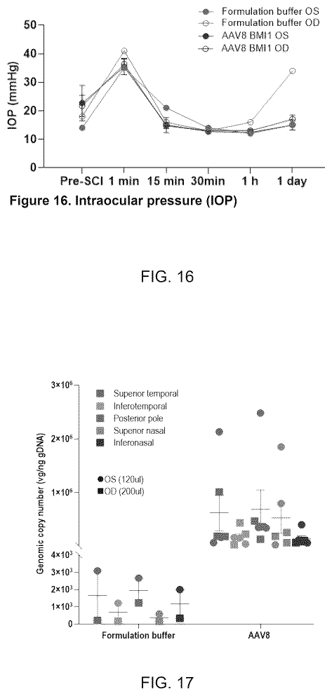

To confirm ocular biodistribution of an AAV8 vector, four 3-month-old Yucatan minipigs were placed under anesthesia and injected with 1¹² vector genomes/eye via SCI with the OCLX-SCDD alpha prototype. Right eyes were treated with 120 μL, and left eyes were treated with 200 μL using a sterile OCLX-SCDD device to determine if volume affects IOP or biodistribution. A control pig was injected with formulation buffer at the same volumes. Intraocular pressures were measured prior to injection, one minute, 15 minutes, 1 hour, and one day after injection (FIG. 16). One day after SC injection (SCI), animals were euthanized according to the approved IACUC protocol. Eyes were snap frozen in liquid nitrogen and subsequently dissected on ice. Punch biopsies were taken of combined retina and RPE/choroid in the posterior pole (5 mm temporal to the optic nerve) and four quadrants. Ocular tissues were then homogenized, and DNA was extracted using commercial kits (QIAGEN). Then qPCR was performed with primers to the AAV8 genome.

Results indicated significantly higher AAV8 vgs in the AAV8-treated eyes compared to formulation buffer-treated eyes in the posterior pole as well as all four quadrants (FIG. 17). There was no significant difference in ocular biodistribution between the 120 μl and 200 μl treated groups. IOP normalized by 15 minutes.

OCLX-SCDD successfully delivers potent and infectious AAV8 to the retina and RPE/choroid at both the 120 μL and 200 μL levels throughout the eye, including the posterior pole.

### Example 12

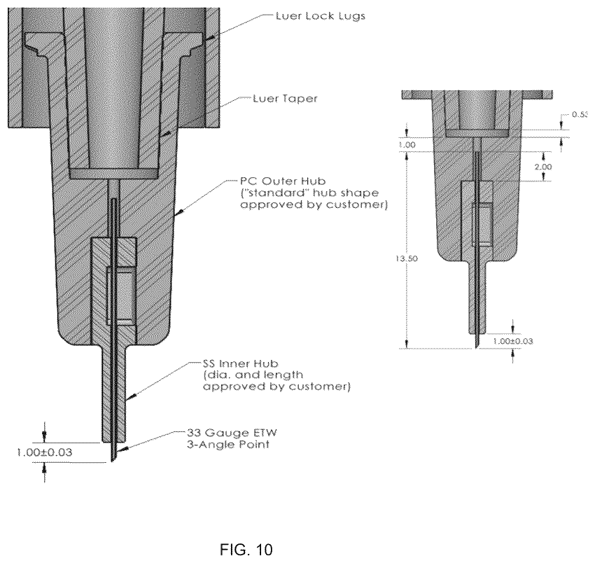

In this set of experiments, tensile strength was measured for the connection between the needle and injector hub as well as inner hub to Luer hub (FIG. 10) using a TA Texture analyzer and 100 kg load cell. A total of 7 SCDD units were tested for each joint location with results summarized in Table 1. With a design specification of tensile strength ≥11N (based on ISO 7864:2016 for a 33 g needle), results were well above requirement for both needle to hub and inner hub to Luer hub.

TABLE 1
Tensile strength results of key joint locations in alpha
prototype (all greater than the 11N threshold requirement)

| Joint | Tensile strength (N) | n |
|---|---|---|
| Needle to hub | 49.4 ± 10.6 | 7 |
| Inner hub to Luer hub | 124.5 ± 18.5 | 7 |

### Example 13

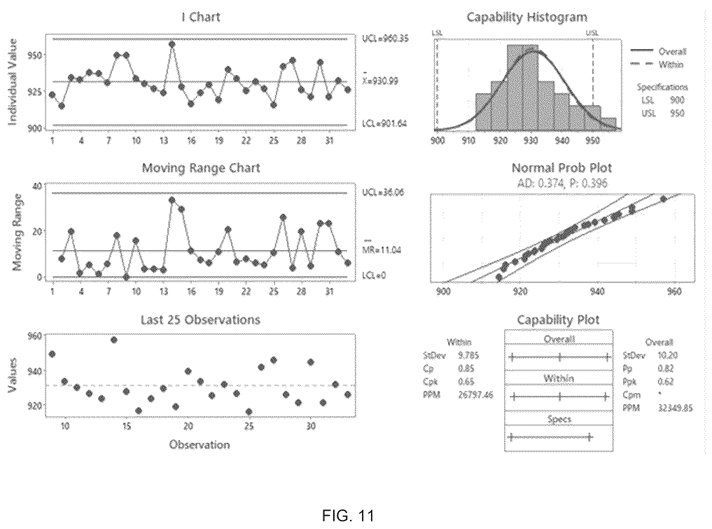

The Process Capability Sixpack Report for exposed needle length in OCLX-SCDD (FIG. 11) provides an analysis of our consistency and capability in meeting specified length requirements measured with Micro-vu (Windsor, CA) equipment. This report includes: (1) The I Chart (Individuals Chart): This chart tracks individual needle lengths across production runs, allowing us to identify any shifts or trends in the manufacturing process; (2) The Moving Range (MR) Chart: The MR Chart shows the variation between consecutive needle lengths, reflecting process variability on a smaller scale. Consistent, low-range values between points suggest a controlled process; (3) Last 25 Observations: This section provides a quick snapshot of the most recent needle length measurements, offering a real-time view of process performance; (4) Capability Histogram: The histogram illustrates the distribution of needle lengths relative to the specified target and tolerance limits. A centered, narrow distribution within the tolerance range indicates that the process reliably produces needles at the desired length; (5) Normal Probability Plot: This plot assesses the normality of needle length data, which is crucial for accurate capability analysis. Data points fall along a straight line, suggesting the lengths follow a normal distribution, which is ideal for applying statistical capability indices; and (6) Capability Plot: The Capability Plot, including indices such as Cp and Cpk, quantifies how well the manufacturing process meets the specified needle length tolerance. Cp and Cpk below 1 suggest that adjustments may be needed to improve accuracy or reduce variability.

Overall, this Process Capability Sixpack Report provides an in-depth view of the injector needle length production process, identifying areas of stability and pinpointing any potential need for process improvements to ensure precise, reliable needle length control.

In an embodiment the needle opening is circular. In another embodiment, the needle opening is oval, square, rectangular, hexagonal, triangular, or such shape as works best to inject the therapeutic into the eye.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present invention, which is defined solely by the claims. Accordingly, the present invention is not limited to that precisely as shown and described.

1. A needle holding unit including:
(a) a needle with a length of 700 to 1100 μm;
(b) a needle base attached to the needle;
(c) a needle hub attached to the needle base that is made of a suitably rigid material, wherein;
(d) the top surface of the needle is a contoured surface extending from a high point to a low point with an angle theta Θ between 22 degrees and 45 degrees.

2. The needle holding unit of claim 1 wherein the needle has a length of 900 to 1000 μm.

3. The needle holding unit of claim 1, wherein the needle has a length of 925±30 μm.

4. The needle holding unit of claim 1, wherein the needle is 29 to 33 gauge.

5. The needle holding unit of claim 1, wherein the needle base has a height of 4 mm.

6. The needle holding unit of claim 1, wherein the needle base is made of silicone.

7. The needle holding unit of claim 1, wherein the needle hub is made of a metal or a plastic.

8. The needle holding unit of claim 1, wherein the contoured surface has an angle relative to a vertical back wall of between 22 and 30 degrees.

9. The needle holding unit of claim 1, wherein the contoured surface has an angle relative to a vertical back wall of between 35 and 40 degrees.

10. The needle holding unit of claim 1, wherein the opening of the needle is circular, oval or oblong.

11. The needle holding unit of claim 1, wherein the needle is made of one or more of stainless steel, tungsten or silicon.

12. The needle holding unit of claim 1 wherein the needle hub includes a locking unit to lock the needle hub to a syringe.

13. The needle holding unit of claim 1 wherein the contoured surface has an angle relative to a vertical back wall of between 22 and 45 degrees.

14. The needle holding unit of claim 12 wherein the locking unit is configured to engage a Luer lock on the syringe.

15. The needle holding unit of claim 12 wherein the locking unit is configured to engage a Catheter lock on the syringe.

16. The needle holding unit of claim 1 wherein the needle includes an opening in the contoured cutting surface with the opening connecting to a channel in the needle that allows fluid to flow.

17. The needle holding unit of claim 1 wherein the lower point of the contoured cutting surface is connected to a lower portion by an offset.

18. The needle holding unit of claim 1 wherein the offset is angled towards the backwall and connects to the low point of the contoured cutting surface.

19. The needle holding unit of claim 1 wherein the dead space volume is either, 28 μl, 29 μl, 30 μl, 31 μl, 32 μl, 33 μl, 34 μl, 35 μl, 36 μl, 37 μl, 38 μl, 39 μl, 40 μl, 41 μl, 42 μl or 43 μl.

20. The needle holding unit of claim 1, wherein each needle holding unit is individually packaged and labeled.
